(19) United States
(12) Patent Application Publication
(10) Pub. No.: US 2012/0059466 A1
(43) Pub. Date: Mar. 8, 2012

(54) INTRAOCULAR LENSES WITH INTERLENTICULAR OPACIFICATION RESISTANCE

(76) Inventors: Chi-Chun Tsai, Fort Worth, TX (US); Brett E. Thomes, Arlington, TX (US); Stephen J. Van Noy, Southlake, TX (US); Atsmon Shahar, Cresson, TX (US)

(21) Appl. No.: 13/195,883

(22) Filed: Aug. 2, 2011

Related U.S. Application Data
(63) Continuation-in-part of application No. 12/874,863, filed on Sep. 2, 2010.

(51) Int. Cl. A61F 2/16; B05D 7/00

(52) U.S. Cl. 623/6.62; 427/2.24

(57) ABSTRACT

The present invention is directed to an intraocular lens, an intraocular lens system and a method of producing and/or implanting the lens or system in an eye wherein at least one intraocular lens includes a coating that aids in resisting opacification (e.g., posterior capsule opacification (PCO), interlenticular opacification (ILO) or the like). The material of the coating is preferably hydrophilic or super-hydrophobic.

### CROSS REFERENCE TO RELATED APPLICATION

This application is a continuation-in-part (CIP) of co-pending U.S. application Ser. No. 12/874,863, filed Sep. 2, 2010, priority of which is claimed under 35 U.S.C. §120, the contents of which are incorporated herein by to reference. This application also claims priority under 35 U.S.C. §119 to U.S. Provisional Patent Application Ser. No. 61/239,974, filed Sep. 4, 2009, the entire contents of which are incorporated herein by reference.

### TECHNICAL FIELD OF THE INVENTION

The present invention relates to an intraocular lens, an intraocular lens system and a method of producing and/or implanting the lens or system in an eye wherein at least one intraocular lens includes a coating that aids in resisting opacification such and posterior capsule opacification (PCO) or interlenticular opacification (ILO).

### BACKGROUND OF THE INVENTION

The human eye functions to provide vision by transmitting and refracting light through a clear outer portion called the cornea, and further focusing the image by way of a lens onto the retina at the back of the eye. The quality of the focused image depends on many factors including the size, shape and length of the eye, and the shape and transparency of the cornea and lens.

When trauma, age, disease or other malady cause an individual's natural crystalline lens to become less transparent, vision deteriorates because of the diminished light which can be transmitted to the retina. This deficiency in the lens of the eye is often referred to as a cataract. The treatment for this condition is surgical removal of the natural crystalline lens and implantation of an intraocular lens (IOL).

While early IOLs were made from hard plastic, such as polymethylmethacrylate (PMMA), soft, foldable IOLs made from acrylate based material have become increasingly popular because of the ability to fold or roll these soft lenses and insert them through a smaller incision. Such acrylate based lenses are particularly desirable because they exhibit excellent folding and unfolding characteristics during and upon implantation within the eye. Such acrylate lenses also exhibit desired biocompatibility characteristics. However, they can be susceptible to posterior capsule opacification (PCO) after implantation.

While typical procedures involve the implantation of only one lens in an eye, there are multiple situations where it is desirable to have a second or two lenses implanted. As one example, dual optic accommodative lenses have been developed to improve the focal range of IOLs. As another example, it may be desirable to, after insertion of a first IOL, implant a second IOL, referred to as piggyback lenses, to improve visual performance.

While such two lens systems can improve visual performance, recent articles have suggested that various types of these lens systems may be susceptible to the development of interlenticular opacification (ILO). Such articles include: Gayton J L, Apple D J, Peng Q, et al., Interlenticular Opacification: A Clinicopathological Correction of a New Complication of Piggyback Posterior Chamber Intraocular Lenses, J. Cataract Refract. Surg., 2000; Eleftheriadis H, Marcantonio J, et al., Interlenticular Opacification in Piggyback AcrySof Intraocular Lenses: Explanation Technique and Laboratory Investigations, Br. J. Ophthalmol. 2001, July 85(7):830-836; and Werner L., Mamalis N., et al., Interlenticular Opacification: Dual-Optic Versus Piggyback Intraocular Lenses, J. Cataract Refract. Surg. 2006, 32:655-661. At least one of these articles suggests that acrylate based two lens systems are susceptible to ILO formation.

In view of the above, it would be quite desirable to provide an intraocular lens, particularly a two lens system, that inhibits opacification such as PCO or ILO that might otherwise occur.

### SUMMARY OF THE INVENTION

Accordingly, there is disclosed an intraocular lens comprised of a body and a coating disposed upon the body. The body is formed of a hydrophobic material. The body defines an outer surface. The coating disposed on a region of the outer surface. The coating is formed of a hydrophilic material or a super-hydrophobic material. The body and coating cooperatively form the intraocular lens. The coating of the intraocular lens is configured to prevent opacification that might otherwise occur.

In preferred embodiments the intraocular lens can have various characteristics. The hydrophobic material of the body can be an acrylate based material. The coating can be formed of a silicone based material. The hydrophobic material can have a contact angle that is at least 40 degrees, more preferably at least 50 degrees but no greater than 85 degrees. The super-hydrophobic material can have a contact angle that is at least 90 degrees. The hydrophilic material can have contact angle that is no greater than 50 degrees and more preferably no greater than 35 degrees.

There is also disclosed a method of producing and/or implanting a coated intraocular lens. According to the method, an intraocular lens having a body defining an outer surface is provided. The body is formed of a hydrophobic material. A coating is disposed upon a region of the outer surface of the body to form the coated intraocular lens. The coating is formed of a hydrophilic material or a super-hydrophobic material. The coated intraocular lens is then implanted within an eye wherein the coating of the intraocular lens is configured to prevent opacification that might otherwise occur. The coated intraocular lens may have any of the characteristics of the lenses described herein.

### BRIEF DESCRIPTION OF THE DRAWINGS

FIG. 1 is a sectional view of a pair of exemplary intraocular lenses that are arranged to form an intraocular lens system in accordance with an aspect of the present invention.

FIG. 2 is a sectional view of a pair of exemplary intraocular lenses are arranged to form an alternative intraocular lens system in accordance with an aspect of the present invention.

FIG. 3 is a front view of an exemplary intraocular lens in accordance with an aspect of the present invention.

FIG. 4 is a sectional view of an exemplary piggyback lens system in accordance with the present invention.

FIG. 5 is a sectional view of an exemplary dual optic accommodative lens system in accordance with the present invention.

### DETAILED DESCRIPTION OF THE INVENTION

The present invention is predicated upon the provision of at least one intraocular lens (IOL) and preferably two IOLs that have a coating for aiding in the prevention of opacification, particularly interlenticular lens opacification (ILO). The IOL[s] typically form an intraocular lens system such as a dual optic or piggyback lens system. The coating is typically formed of a hydrophilic or super-hydrophobic material for aiding in the resistance or prevention of ILO.

Unless otherwise specifically stated, percentages of materials as used herein are weight percentages (w/w).

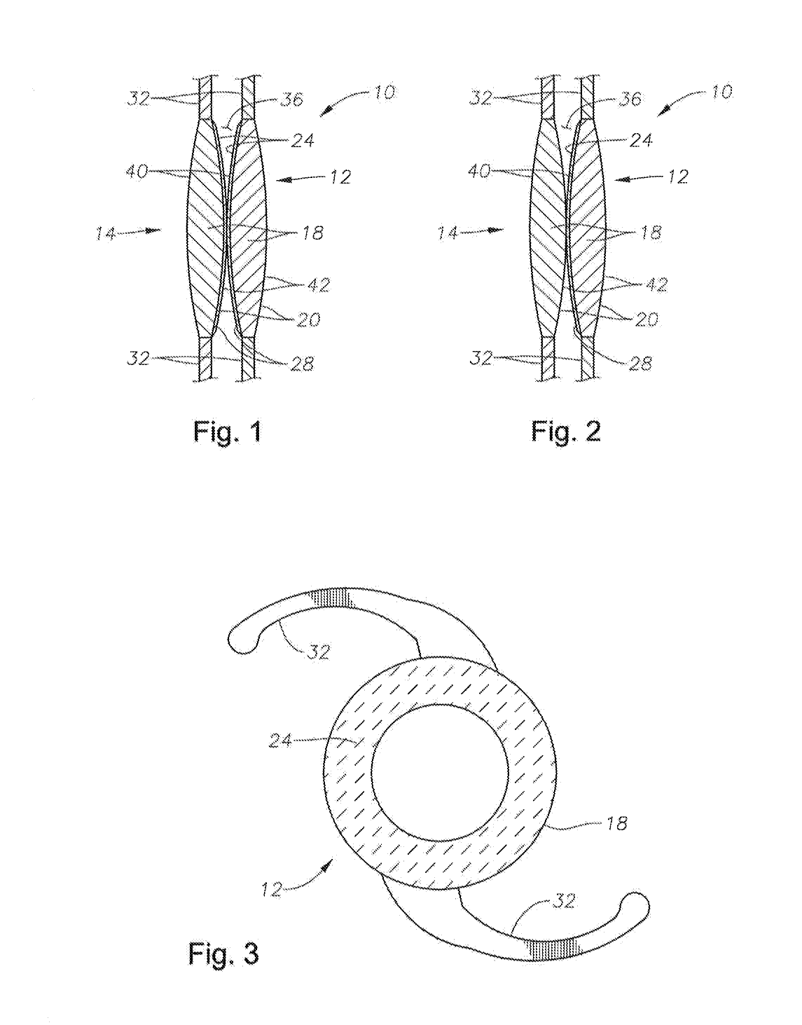

FIG. 1 illustrates an exemplary intraocular lens system 10 in accordance with an aspect of the present invention. The system 10 includes a first intraocular lens 12 and a second intraocular lens 14. As used herein, the terms “first” and “second” as they are used to indicate a lens of the system are merely used to indicate one of the lenses as opposed to the other. These terms are not intended to suggest any order such as order of implantation, unless otherwise specifically stated.

Each of the lenses 12, 14 includes a body 18 defining an outer surface 20 and a coating 24 disposed upon a region 28 of that outer surface 20. The coatings 24 of the lenses 12, 14 can aid in the prevention of ILO as is discussed further below. Each of the lenses 12, 14 also includes haptics 32 extending outwardly from the bodies 18 of the lenses 12, 14.

Each coating 24 of each of the lenses 12, 14 faces and opposes the outer surface 20 of the other of the lenses 12, 14. This is particularly the case after both lenses have been implanted within an eye. The intraocular lenses 12, 14 define an interlenticular space 36 therebetween and the coatings 24 of the lenses 12, 14 are both located directly adjacent and at least partially define the interlenticular space 36.

In the embodiment shown in FIG. 1, each of the lenses 12, 14 has its own coating 24. However, it is contemplated that only one of the lenses may have a coating while the other lens may be uncoated. This configuration is shown in FIG. 2. This may be the case, for example, when the intraocular system includes a set of piggyback lenses for which a first uncoated lens has already been implanted and a second coated lens is implanted as an adjustment to the first lens.

In the embodiment of FIG. 1, the coating 24 of each lens 12, 14 is disposed upon a region 28 of the body 18 and more particularly is disposed only upon one of two opposing sides 40, 42 of the body 18. It is contemplated, however, that the coating may be disposed upon other regions of the body or the entirety of the body of the lens. The term “region” as used herein is intended to mean only a portion of the body. However, the suggestion that the coating covers or is disposed upon a region of the outer surface of the body is not intended to restrict the coating from being located on other portions of the body unless it is specifically stated that the coating is only disposed upon that region.

In instances where the coating is selectively disposed upon only a region of the IOL, it is generally preferred that the region be a substantial portion of the outer surface of the body of the IOL. Preferably, that substantial portion is at least 20%, more preferably at least 40% and even possibly at least 60% of the outer surface of the body. The substantial portion is typically less than 90% and more typically less than 80% of the outer surface of the body. The aforementioned percentages are taken as percentages of total surface area of the body. The outer surface of the body is considered exclusive of any outer surface area of the haptics. Of course, the haptics may also be coated, but are not considered part of the body.

In one preferred embodiment, the coating is formed as a ring about only a peripheral region of the IOL body as shown in FIG. 3. In such an embodiment, the peripheral region may be on only one side of the IOL or on both sides. It is contemplated that a second IOL in a system according to the present invention could have a ring shaped coating that is configured to oppose and face the ring shaped coating of FIG. 3 or such second IOL may have an alternative coating shape such as a coating covering one entire side of its body.

While the coatings of the present invention are particularly desirable for dual lens systems, the at least one coated lens discussed above could be a singular lens that is not intended as being part of a dual lens system. Many standard singular IOLs can benefit from the coating described herein for the prevention of opacification, particularly PCO. Such lenses can include a monofocal lens, a multi-focal lens, a diffractive lens, a toric lens and/or an accommodative lens. The skilled artisan will understand that such lenses may be partially or more fully coated as described relative to the lenses in FIGS. 1-3.

The body, the haptics or both of any of the intraocular lenses according to the present invention are preferably formed of a hydrophobic material. Such hydrophobic material will typically have a contact angle that is no greater than 90 degrees, more typically no greater than 85 degrees and even possibly no greater than 80 degrees. Such material will also typically have a contact angle that is at least 50 degrees and more typically at least 60 degrees and even possibly at least 65 degrees. Unless stated otherwise, contact angles for the materials of the present invention are determined in accordance with Young's equation as discussed in Physical Chemistry of Surfaces (sixth edition), Adamson, Arthur W. et al. Chapter X, pgs. 352-354.

The material of the body, the haptics or both is preferably an acrylate based material. Acrylate based materials are defined as having a substantial portion of acrylate monomers, which are preferably of formulation 1 below:

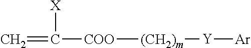

wherein: X is H or CH₃;
m is 0-10;
Y is nothing, O, S, or NR wherein R is H, CH₃, CₙH₂ₙ₊₁(n=1-10), iso-OC₃ H₇, C₆H₅, or CH₂C₆H₅;
Ar is any aromatic ring which can be unsubstituted or substituted with CH₃, C₂H₅, n-C₃H₇, iso-C₃H₇, OCH₃, C₆H₁₁, C₆H₅, or CH₂C₆H₅;

Suitable monomers of structure (I) include, but are not limited to: 2-ethylphenoxy methacrylate; 2-ethylphenoxy acrylate; 2-ethylthiophenyl methacrylate; 2-ethylthiophenyl acrylate; 2-ethylaminophenyl methacrylate; 2-ethylaminophenyl acrylate; phenyl methacrylate; phenyl acrylate; benzyl methacrylate; benzyl acrylate; 2-phenylethyl methacrylate; 2-phenylethyl acrylate; 3-phenylpropyl methacrylate; 3-phenylpropyl acrylate; 4-phenylbutyl methacrylate; 4-phenylbutyl acrylate; 4-methylphenyl methacrylate; 4-methylphenyl acrylate; 4-methylbenzyl methacrylate; 4-methylbenzyl acrylate; 2-2-methylphenylethyl methacrylate; 2-2-methylphenylethyl acrylate; 2-3-methylphenylethyl methacrylate; 2-3-methylphenylethyl acrylate; 24-methylphenylethyl methacrylate; 2-4-methylphenylethyl acrylate; 2-(4-propylphenyl)ethyl methacrylate; 2-(4-propylphenyl)ethyl acrylate; 2-(4-(1-methylethyl)phenyl)ethyl methacrylate; 2-(4-(1-methylethyl)phenyl)ethyl acrylate; 2-(4-methoxyphenyl)ethyl methacrylate; 2-(4-methoxyphenyl)ethyl acrylate; 2-(4-cyclohexylphenyl)ethyl methacrylate; 2-(4-cyclohexylphenyl)ethyl acrylate; 2-(2-chlorophenyl)ethyl methacrylate; 2-(2-chlorophenyl)ethyl acrylate; 2-(3-chlorophenyl)ethyl methacrylate; 2-(3-chlorophenyl)ethyl acrylate; 2-(4-chlorophenyl)ethyl methacrylate; 2-(4-chlorophenyl)ethyl acrylate; 2-(4-bromophenyl)ethyl methacrylate; 2-(4-bromophenyl)ethyl acrylate; 2-(3-phenylphenyl)ethyl methacrylate; 2-(3-phenylphenyl)ethyl acrylate; 2-(4-phenylphenyl)ethyl methacrylate; 2-(4-phenylphenyl)ethyl acrylate; 2-(4-benzylphenyl)ethyl methacrylate; and 2-(4-benzylphenyl)ethyl acrylate, and the like.

It is contemplated that the first and second IOLs of a system can be formed of substantially identical material, but may be formed of different materials. Preferably, the material of both IOLs of the system are acrylate based, however, it is possible for one to be acrylate based while another may be formed of a different material (e.g., a silicone based material). In such circumstances, the acrylate based IOL will typically include a coating according to the present invention while the other IOL of different material may or may not include a coating.

The material of the body and/or haptics is typically formed from at least 30%, more typically at least 70% and even possibly at least 95% acrylate monomers. The material of the body and/or haptics is typically formed from no greater than about 99.9% acrylate monomers. These acrylate based materials are typically mixed with a curing agent and/or a polymerization initiator so that the materials may be cured to form the IOLs. As such, it will be understood that these monomers are linked to form polymers in the finished IOLs. Examples of acrylate-based lenses are, without limitation, described in U.S. Pat. Nos. 5,922,821; 6,313,187; 6,353,069; and 6,703,466, all of which are fully incorporated herein by reference for all purposes.

The coating is preferably formed of a hydrophilic material or a super-hydrophobic material. A suitable hydrophilic material will typically have a contact angle that is no greater than 50 degrees, more typically no greater than 45 degrees and even possibly no greater than 35 degrees. Such material will typically have a contact angle that is at least 5 degrees.

A hydrophilic coating can also be formed of a hydrogel material. In such an embodiment, functionalized hydrogel precursors of hydrogel materials such as polyacrylic acid (PAA), polyvinyl acetate (PVA), polyvinyl pyrrolidone (PVP), polyethylene glycol (PEG), polyether imide (PEI), combinations thereof or the like may be coated upon the outer surface of the IOL body. The precursors can then be cross-linked by ultraviolet and/or visible light, plasma, radiation, heat energy or the like to form the coating of hydrogel material.

A suitable super-hydrophobic material for the coating will typically have a contact angle that is at least 90 degrees, more typically at least 100 degrees and even more possibly at least 130 degrees. Such material will typically have a contact angle no greater than 177 degrees.

When the coating is formed of a super-hydrophobic material, silicone based materials are typically quite desirable. Silicone based materials are those materials that include a substantial portion of silicon or silicon monomers (e.g., silane or siloxane). When silicone based, the material of the coating typically is formed from at least 30%, more typically at least 60% and even possibly at least 80% silicone monomers. In such embodiment, the material of the coating is typically formed from no greater than about 99.9% silicone monomers. Examples of silicone materials are, without limitation, described in U.S. Pat. Nos. 5,420,213; 5,494,946; 7,033,391; and 7,071,244, all of which are fully incorporated herein by reference for all purposes.

Silicone based coatings can be formed upon the body of the IOL using various techniques. In one embodiment, silicon monomers (e.g., silane or siloxane monomers) can be coated on the outer surface of the body by plasma deposition or polymerization onto the surface of the body. In another embodiment, plasma treatment (e.g., oxygen or water plasma treatment) can be employed to introduce hydroxyl groups onto the outer surface of the IOL body followed by a silanization treatment. In yet another embodiment, a surface modifying agent containing silicone block copolymer can be blended with the acrylate material prior to casting and curing of the IOL.

As an alternative to silicone, super-hydrophobic materials with even greater hydrophobicity (e.g., contact angles of at least 130 degrees) may be used. These super-hydrophobic coatings can be formed using continuous or, more preferably, modulated plasma deposition/polymerization treatment of perfluorocarbons monomers, which can then be cross-linked to form a polytetrafluoroehtylene (PTFE) coating. As an alternative, benzene moieties can be attached to the IOL body outer surface by direct fluorination to form a super-hydrophobic coating. As another alternative, plasma treatment (e.g., oxygen or water plasma treatment) can be used to introduce hydroxyl groups onto the outer surface of the IOL body followed by a fluorinated silanization treatment.

As an alternative or addition to a hydrophilic or super-hydrophobic coating, it is contemplated that a coating may be formed of bioactive agents. As one example, natural or synthetic molecules that modulate or inhibit protein adsorption and/or cell adhesion can be attached to the outer surface of the body to form a modified surface coating (e.g., a modified surface that preferentially adsorb serum albumin). As another example, pharmacological agents such as immunosuppressants, mTOR inhibitors or the like can be attached or otherwise coated on the outer surface of the IOL body to form a coating that prohibits or inhibits lens epithelial cell (LEC) growth. It is also contemplated that a coating may only cover a peripheral region (e.g., a peripheral edge) of the lens body and, for example, may form a ring about the lens body and/or may extend radially outwardly from the peripheral region. Still further, it is contemplated that the coating may be formed as a separate solid film (e.g., an annular disc shape film) that is then disposed over the surface of the lens body and preferably attached (e.g., adhered) thereto.

Implantation

Lens systems of the present invention can be implanted in the eye according various protocols. Of course, a singular intraocular lens can be implanted through an incision into the capsular bag or other region inside the eye. However, protocols for insertion of a dual lens system may vary. Typically a first lens is implanted followed by a second lens. It is contemplated, however, that two lenses may be implanted at least partially simultaneously. Both lenses may be implanted in the capsular bag or one may be located in the capsular bag while the other is outside of the capsular bag.

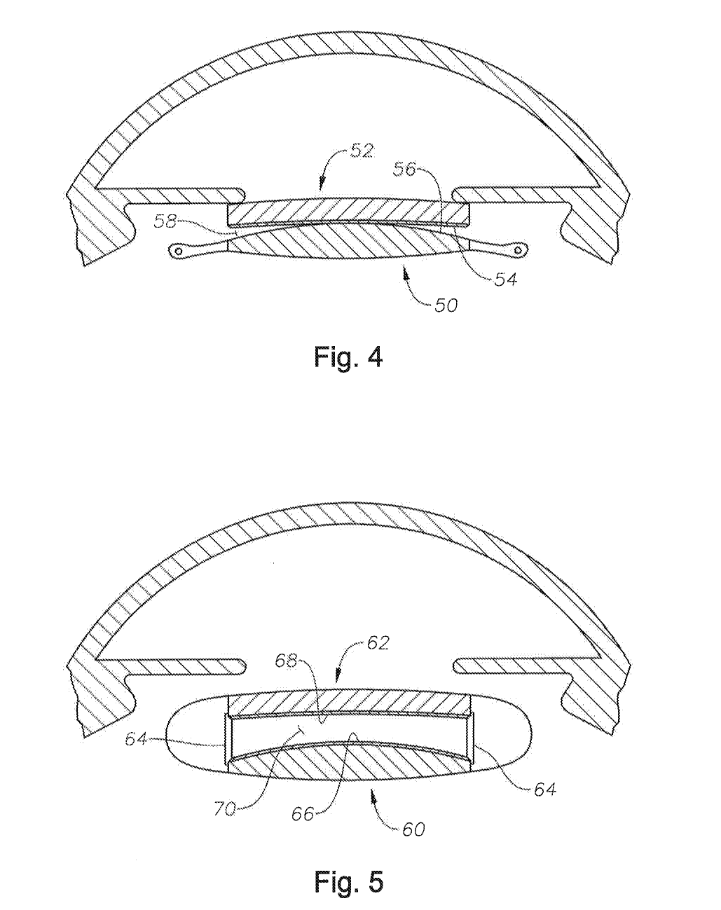

In one preferred embodiment, a first lens is implanted in the capsular bag and then, upon discovery that the first lens is not providing the desired visual performance, a second lens is implanted in the sulces of the eye. Such lenses are typically referred to as piggy-back lenses. As example of such lenses are shown in FIG. 4. As can be seen, a first lens 50 is disposed in the capsular bag and is without a coating. However, a second lens 52, which has been implanted later in the sulces does include a coating 54 in accordance with the present invention. Generally, for piggy-hack lens systems, the lens implanted in the sulces or the second lens implanted will be the only lens to include a coating since the lens in the capsular bag will have been implanted without the knowledge that a second lens would necessarily be implanted. Of course, it would be possible for the first implanted lens 50 (i.e., the lens in the capsular bag) to also include a coating, particularly if there is a likelihood that a second piggyback lens will be implanted later. In the embodiment shown, the coating 54 is in opposing facing relation to an outer side surface 56 of the first lens 50 and directly adjacent an interlenticular space 58 between the lenses.

In another preferred embodiment, a first lens is implanted in the capsular bag and then a second lens is implanted in the capsular hag and connected to the first lens to form a dual optic intraocular lens system (e.g., an accommodative system). As can be seen in FIG. 5, a first lens 60 having positive power is implanted and a second lens 62 having negative power is implanted. They are then attached to each other with attachment members 64 (e.g., interlocking haptics or other members) to form a dual optic accommodative intraocular lens system. As can also be seen, both of the lenses 60 and 62 having coatings 66, 68 on only one side of the lenses 60, 62 and those coatings 66, 68 are in opposing facing relation to each other and adjacent an intralenticular space 70.

### Example

A hexamethyldisiloxane (HMDSO) RF plasma polymer film is deposited upon a plurality of IOLs. Initially, approximately 5 grams of HMDSO monomer is added to a 50-mL round bottom flask containing a magnetic stirring bar. The flask is then attached to a glass vacuum take-off adapter which is connected to a plasma chamber with a stainless steel gas line. Vapor flow rate is regulated through a metering valve located on the gas line. The flask is placed on a heating mantle which is seated on a stirring plate. The mantle is used to offset cooling of the flask due to expansion of the contents under high vacuum. This aids in maintaining a steady flow rate throughout the deposition process. Next, IOLs are placed in the center of the plasma chamber. The chamber is then sealed and vacuumed down to less than 10 mTorr. Next, the metering valve is opened for several minutes to allow the flask and HMDSO monomer to fully degas. With degassing complete, the valve is closed and the chamber is again vacuumed down to less than 10 mTorr. The metering valve is then opened fully and the chamber pressure is set to 100 mTorr. Pressure in the plasma chamber is tightly regulated with a pressure controller that is attached to a throttling valve. With the pressure stabilized, the RF unit is sent to 220 watts and switched on. This starts the deposition process which continues for five minutes. Next, the RF unit is switched off, the metering valve is closed and the chamber is evacuated to less than 10 mTorr. Finally, the chamber is returned to atmospheric pressure with argon gas and the coated lenses are removed.

The entire contents of all cited references in this disclosure are specifically incorporated herein by reference. Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

Other embodiments of the present invention will be apparent to those skilled in the art from consideration of the present specification and practice of the present invention disclosed herein. It is intended that the present specification and examples be considered as exemplary only with a true scope and spirit of the invention being indicated by the following claims and equivalents thereof.

We claim:

1. An intraocular lens comprising:
a body formed of a hydrophobic material, the body defining an outer surface; and
a coating disposed on a region of the outer surface of the body, the coating being formed of a hydrophilic material or a super-hydrophobic material wherein:
i) the body and coating cooperatively form the intraocular lens; and
ii) the coating of the intraocular lens is configured to prevent opacification that might otherwise occur.

2. An intraocular lens as in claim 1 wherein the coating is disposed only on the region of the body leaving a substantial portion of the body uncovered by the coating.

3. An intraocular lens as in claim 2 wherein the substantial portion is at least 40% of the surface of the body.

4. An intraocular lens as in claim 2 wherein the substantial portion is at least 60% of the surface of the body.

5. An intraocular lens as in claim 1 wherein the hydrophobic material is an acrylate based material.

6. An intraocular lens as in claim 1 wherein the coating is formed of a silicone based material.

7. An intraocular lens as in claim 6 wherein the coating is formed from HMDSO.

8. An intraocular lens as in claim 1 wherein the hydrophobic material has a contact angle that is at least 40 degrees but no greater than 85 degrees, the super-hydrophobic material has a contact angle that is at least 90 degrees, the hydrophilic material has a contact angle that is no greater than 50 degrees.

9. An intraocular lens as in claim 8 wherein the hydrophobic material has a contact angle that is at least 50 degrees.

10. An intraocular lens as in claim 9 wherein the hydrophilic material has a contact angle that is no greater than 35 degrees

11. A method of producing and/or implanting a coated intraocular lens, the method comprising:
providing an intraocular lens having a body defining an outer surface, the body being formed of a hydrophobic material; and
disposing a coating upon a region of the outer surface of the body to form the coated intraocular lens, the coating being formed of a hydrophilic material or a super-hydrophobic material;
implanting the intraocular lens within an eye, wherein:
i) the coating of the intraocular lens is configure to prevent opacification that might otherwise occur.

12. An intraocular lens as in claim 11 wherein the coating is disposed only on the region of the body leaving a substantial portion of the body uncovered by the coating.

13. An intraocular lens as in claim 12 wherein the substantial portion is at least 40% of the surface of the body.

14. An intraocular lens as in claim 12 wherein the substantial portion is at least 60% of the surface of the body.

15. A method as in claim 11 wherein the hydrophobic material is an acrylate material.

16. A method as in claim 15 wherein the coating if formed of a silicone material.

17. A method as in claim 16 wherein the coating is formed from HMDSO.

18. A method as in claim 11 wherein the hydrophobic material has a contact angle that is at least 40 degrees but no greater than 85 degrees, the super-hydrophobic material has a contact angle that is at least 90 degrees, the hydrophilic material has a contact angle that is no greater than 50 degrees.

19. An intraocular lens as in claim 18 wherein the hydrophobic material has a contact angle that is at least 50 degrees.

20. An intraocular lens as in claim 19 wherein the hydrophilic material has a contact angle that is no greater than 35 degrees
